(19) United States
(12) Patent Application Publication
(10) Pub. No.: US 2023/0069687 A1
(43) Pub. Date: Mar. 2, 2023

(54) VALVE PLANNING TOOL

(71) Applicant: GYRUS ACMI, INC D/B/A OLYMPUS SURGICAL TECHNOLOGIES AMERICA, Westborough, MA (US)

(72) Inventors: Ryan Ponten, Redmond, WA (US); Jean-Martin Baillargeon, Seattle, WA (US)

(21) Appl. No.: 17/823,241

(22) Filed: Aug. 30, 2022

Related U.S. Application Data
(60) Provisional application No. 63/239,210, filed on Aug. 31, 2021.

(51) Int. Cl. A61M 25/01; A61B 1/005

(52) U.S. Cl. CPC A61M 25/0136; A61B 1/0052

(57) ABSTRACT

Systems, methods, and executable programs for providing lung candidacy information to health care professionals. In an illustrative embodiment, a valve planning tool including a flexible shaft having a distal end and a proximal end, and a plurality of flexible sizing elements configured to be attachable to the flexible shaft. The flexible sizing elements are extendable radially from the flexible shaft when in a deployed state and at least partially foldable around a longitudinal axis of the flexible sizing elements when in a retracted state.

### BACKGROUND

The present disclosure related to a valve planning tool. Currently, passageways are measured using balloons that are inflated using a non-compressible fluid such as saline until the balloon fills the passageway. The amount of fluid placed in the balloon during sizing is then compared to a sizing chart to determine the proper sized valve to seal a given airway. While this process may be very effective at measuring passageways, calibration of the balloon is performed every time to ensure accuracy.

### SUMMARY

The present disclosure provides systems, methods, and executable programs for providing lung candidacy information to health care professionals.

In an illustrative embodiment, a valve planning tool including a flexible shaft having a distal end and a proximal end, and a plurality of flexible sizing elements configured to be attachable to the flexible shaft. The flexible sizing elements are extendable radially from the flexible shaft when in a deployed state and at least partially foldable around a longitudinal axis of the flexible sizing elements when in a retracted state.

In an illustrative embodiment, a system including a scope having a handle and an insertion tube with a working channel and a camera, an image processor configured to be in signal communication with the camera, a display device configured to be in signal communication with the image processor, and a valve planning tool. The valve planning tool includes a flexible shaft having a distal end and a proximal end and a plurality of flexible sizing elements configured to be attachable to the flexible shaft. The flexible sizing elements are extendable radially from the flexible shaft when in a deployed state and at least partially foldable around a longitudinal axis of the flexible sizing elements when in a retracted state.

In an illustrative embodiment, a method including providing a flexible shaft having a distal end and a proximal end; and attaching a plurality of flexible sizing elements to the flexible shaft. The flexible sizing elements are extendable radially from the flexible shaft when in a deployed state; and at least partially foldable around a longitudinal axis of the flexible sizing element when in a retracted state.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 is a perspective view and block diagram of an illustrative bronchoscope system.

FIG. 2 is a perspective view of an illustrative tool for determining airway size.

FIG. 3 is a perspective view of a distal end of the tool of FIG. 2.

FIG. 4 is a perspective view of the distal end of the scope in FIG. 1 with the tool of FIG. 2.

FIG. 5 is a cross-sectional view of the tool of FIG. 3 in a partially collapsed state.

FIG. 6 is a side view of the handle of the scope of FIG. 1 with a valve planning tool received therein.

FIG. 7 is a perspective view of a valve planning tool.

FIG. 8 is a side view of the valve planning tool of FIG. 7 relative to an endobronchial valve in different states.

FIG. 9 is a perspective view of a portion of a valve planning tool.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various disclosed embodiments include illustrative tools, systems, and methods for performing planning before selection and installation of an endobronchial valve.

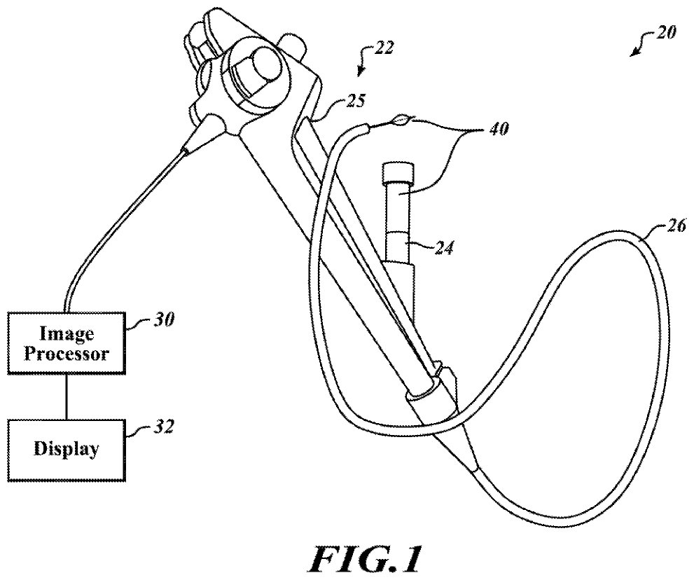

Referring now to FIG. 1, a valve planning system 20 includes a bronchoscope 22 having an insertion tube 26, an image processor 30, and a display device 32. The bronchoscope 22 includes a camera at a distal end of the insertion tube 26 that provides image data to the image processor 30 for generation of images for presentation on the display device 32. A valve planning tool 40 is slidably received within the bronchoscope 22 via a port 24 of a handle 25.

The display device 32 is in wired or wireless signal communication with the signal processor 24. The display device 32 presents images based on information received from the camera of the bronchoscope 22. A diagnostic bronchoscope (e.g., BF-MP190F produced by Olympus®) is an example of the bronchoscope 22. The bronchoscope 22 may include a 3.0 mm distal-end outer diameter with a 1.7 mm working channel, thereby facilitating deep access into the lung. Other types of bronchoscopes may be used such as a bronchoscope with an outer diameter less than 4.2 mm, less than 3.5 mm, less than 3.2 mm, less than 3.0 mm, and the like.

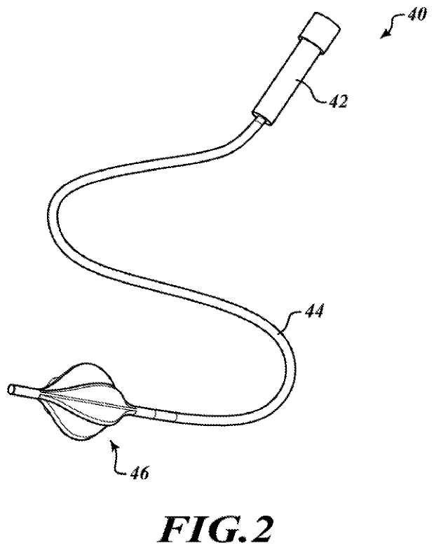

Referring now to FIGS. 2-5, the valve planning tool 40 includes a handle 42, a flexible shaft 44 coupled at a proximal end to a distal end of the handle 42, and a distal sizing tool 46. The distal sizing tool 46 is located at a distal end of the flexible shaft 44. The distal sizing tool 46 includes a base 52 and a plurality of sizing elements 50 that extend radially from the base 52. The base 52 may be attached with a medical grade adhesive to the flexible shaft 44 or may include a lumen configured to receive the flexible shaft 44 with adhesive to secure the two or a pressure fit that keeps the base 52 fixed to the flexible shaft 44.

In various embodiments, a various number of sizing elements 50 may be used depending upon the application. In one embodiment, the distal sizing tool 46 includes six sizing elements 50 spaced equidistant around the base 52. In this embodiment, each of the sizing elements 50 may simulate struts of an endobronchial valve, such as the Spiration® valve system. The sizing elements 50 have an outer edge 56 that has a varying distance from the base 52. The outer edge 56 has a first distance value from the base 52 at a proximal end of the sizing elements 50, a second value from the base 52 at a distal end of the sizing elements 50, and a third value from the base 52 at approximately a halfway point between the proximal and distal ends. The distance of the outer edge 56 from the base 52 decreases from the third distance value as the outer edge 56 transitions to the proximal and distal ends (to the first and second values). A side view of the sizing elements 50 may have a roughly parabolic shape. The third value of the outer edge 56 simulates the widest opening point (sealing section) of a basket or umbrella section of an endobronchial valve. In various embodiments, the sizing elements 50 at the third value may include a visually distinctive marking(s), such as, without limitation, texture, color, or other markings. The visually distinctive markings are visible within the generated and displayed images produced by the camera of the bronchoscope 22.

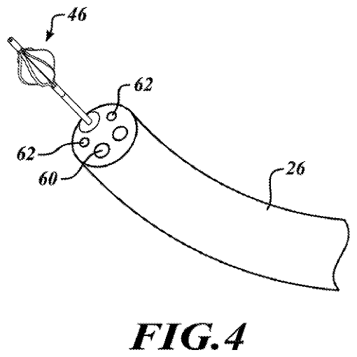

Referring additionally to FIG. 4, the insertion tube 46 includes a working channel 58, lights or fiber-optic light carriers 62, and a distal camera 60. The valve planning tool 40 is received through the port 24 of the handle 25, through the working channel 58 of the insertion tube 26, then out the distal end of the working channel 58. Once the distal sizing tool 46 exits the working channel 58, the sizing elements 50 transition from a collapsed state to an expanded state. The distal sizing tool 46 is moved to a target location within an airway by an operator with aid from images produced by the camera 60 and presented on the display 32.

In various embodiments, the outer edge 56 of the sizing elements 50 are beveled or chamfered along the entire length of the outer edge 56 or just a portion of the outer edge 56. The outer edges 56 are configured to make contact with an inner wall of the working channel 58 causing the sizing elements 50 to be forced circumferentially around the base 52. The outer edges 56 aid in the collapse of the sizing elements 50 as the distal sizing tool 46 is retracted into the working channel 58.

In various embodiments, the sizing elements 50 include elements having different third distance values. The sizing elements 50 may include elements with two or more different third distance values. Each of the differently sized sizing elements 50 simulate different sizes of actual endobronchial valves available for implantation. The sizing elements 50 may be flexible enough to fold when the element makes contact with an airway wall. Thus, if one of the elements is too big for an airway, it will fold, thereby allowing smaller elements to give a better assessment as to the size of the airway.

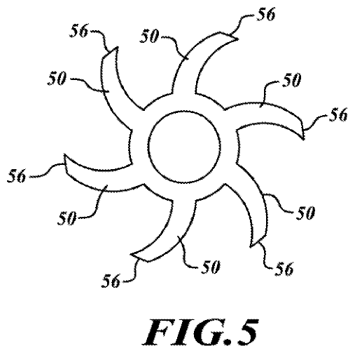

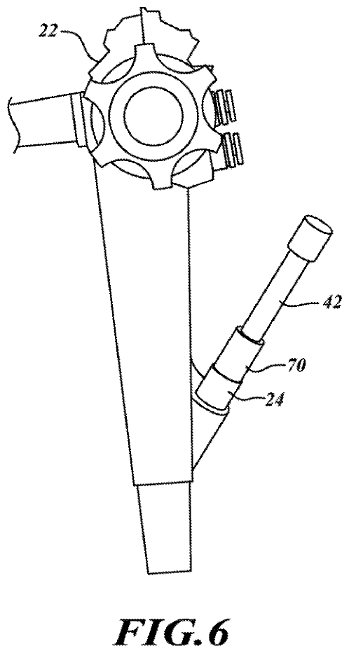

Referring additionally to FIG. 6, the handle 42 of the valve planning tool 40 may be received by a rotation tool 70. The rotation tool 70 is configured to attach to the port 24 and allow rotation of the handle 42 and thus the flexible shaft 44 and the distal sizing tool 46 upon retraction in the proximal direction. The rotation tool 70 may include grooves or pawls in conjunction with corresponding grooves or pawls on an outer surface of the handle 42. The rotation tool 70 is configured to cause rotation, thus taking advantage of the chamfered edges 56 of the sizing elements 50. As such, referring to FIG. 5, as illustrated, the rotational motion created by the proximal motion of the handle 42 passing through the rotation tool 70 may be a counterclockwise motion.

The sizing elements 50 are configured to measure one or more cross-sectional lengths of a structure. The sizing elements 50 may measure one or more diameters of a structure. In various embodiments, the structure is a passageway, such as, without limitation, an airway. In various embodiments, the sizing elements 50 measures a plurality of cross-sectional lengths (e.g., diameter) of a passageway. The sizing elements 50 may measure a passageway to determine which size valve is desirable to seal the passageway. For example, the passageway may measure a length of a passageway from one side to the other despite the passageway being non-circular or irregular in shape. If the passageway is circular, then the cross-sectional length is a diameter.

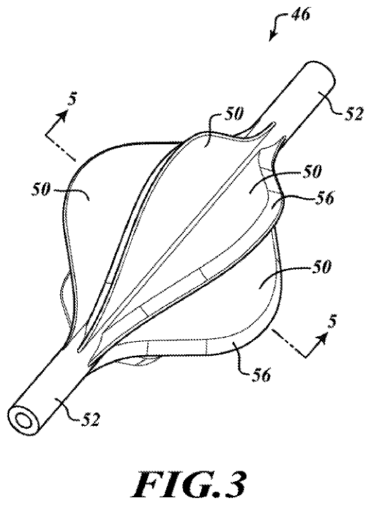

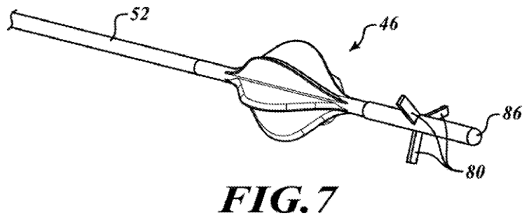

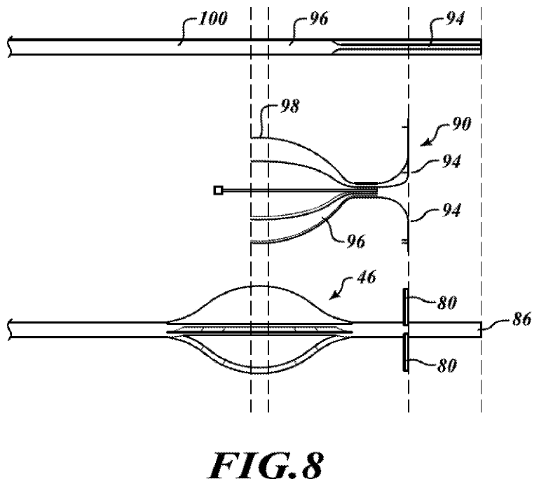

Referring additionally to FIGS. 7 and 8, in various embodiments a valve planning tool 78 includes the distal sizing tool 46 of FIG. 3 and one or more tangs 80 (anchor indicator) that are attached to the distal end of the distal sizing tool 46 or to the shaft 44 received through a lumen of the distal sizing tool 46. The tangs 80 are expandable and retractable. The distal sizing tool 46 and the tangs 80 may have dimensions that mirror features of an endobronchial valve and delivery catheter. The valve planning tool 78 has a dimension (i.e., length) that is equal to a distance of a distal end of a delivery catheter relative to a seal location/line of the valve. The valve planning tool 78 may have a length from the widest cross-sectional diameter of the distal sizing tool 46 (seal location) to a distal tip 86 that simulates a total length that the delivery catheter extends into a passage during valve deployment. The valve planning tool 78 may have a length of about 8 mm or more, about 10 mm or more, about 11 mm or more, about 12 mm or more when measured from a seal area indicator to the tip 86.

When in a deployed state of the tangs 80 extend radially outward from a base. The extended tangs 80 may be sized to simulate anchor position relative to the distal sizing tool 46 as well as the size of the anchors of an endobronchial valve. The tangs 80 may function to determine if the anchors of the valve can open. The tangs 80 may function to measure a minimum size passageway that a valve may seal. In the retracted state the sizing elements 50 and the tangs 80 fold or compress into the working channel 58.

In various embodiments and given by way of overview, the shaft 44 may be rigid, flexible, or has variable flexibility. The shaft 44 may be made of plastic, metal, a bio-compatible material, or a combination thereof.

In various embodiments, the sizing elements 50 and the tangs 80 may be made of a highly flexible, compressible material that is resistant to permanent compression, such as, without limitation, a rubber-type plastic or a silicone rubber.

The tangs 80 may function to measure the passageway for a maximum cross-sectional length of next size down or a minimum cross-sectional length of a next size up valve relative to the cross-sectional length being measured using the sizing elements 50. The tangs 80 may have a cross-sectional length of about 6 mm or more, about 8 mm or more, about 9 mm or more, about 10 mm or more, or about 11 mm or more. Other sizes larger or smaller than those described above may be selected depending upon size of a target lumen.

Referring additionally to FIG. 8, the valve planning tool 78 is shown in the deployed state. The valve planning tool 78 is shown aligned with a valve 81 in a deployed state and a valve delivery catheter 87 with the valve 81 in a delivery state. The valve planning tool 78 includes a seal indicator 79, which is the widest cross-sectional position of the sizing elements 50 of the distal sizing tool 46. If the sizing elements 50 are not lined up directly opposite the base 52, then the seal indicator 79 is the third distance value of one of the sizing elements 50 added to the third distance value of another one of the sizing elements 50. The seal area indicator 79 simulates the proximal end of the valve 81. The seal indicator 79 is shown aligned with a seal location 89 of a corresponding (size wise) valve 81 in an expanded, deployed state and the seal location 89 of the valve 81 when in a compressed, delivery state within a delivery catheter 87. The tangs 80 simulate location of anchors 83 of the valve 81 in the deployed state. The distal tip 86 simulates location of the distal end of the delivery catheter 87.

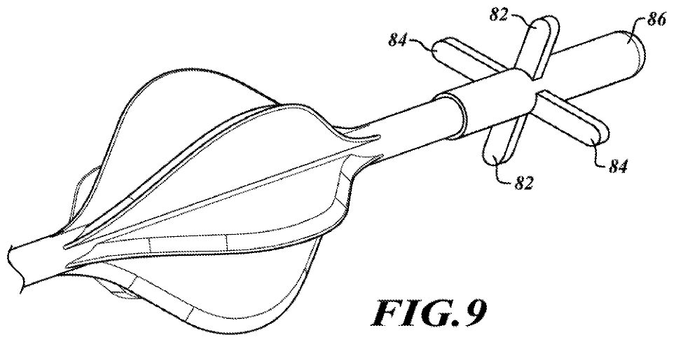

Referring additionally to FIG. 9 in various embodiments, a valve planning tool 90 may have one or more minor tangs 82 and two or more major tangs 84. The tangs 82 and 84 simulate, like the tangs 80 (FIG. 7), anchor location of a corresponding valve. The major tangs 84 and minor tangs 82 may be located apart by any degree interval.

The tangs 82 and 84 may have a length that simulates anchor expansion of an endobronchial valve, thus indicating if the valve anchors can contact the passageway. The one or more tangs 82 and 84 may be resilient so that the tangs move to an open or free state. A longitudinal distance from the tangs 82 and 84 to the widest part of the sizing elements 50 simulate the length of a valve. Also, the tangs 82 and 84 may be used to measure passageway width.

In various embodiments, a kit (not shown) will may include one or more of the valve planning tools having different length and width sizes for simulating each of the actual valves that may be used. The kit includes two or more valve planning tools, three or more valve planning tools, and four or more valve planning tools. When the kit includes more than one valve planning tool the included valve planning tools are all a different size. The kit may include the same number of valve planning tools as there are available valve sizes.

### Embodiments

A. A valve planning tool comprising: a flexible shaft having a distal end and a proximal end; and a plurality of flexible sizing elements configured to be attachable to the flexible shaft, the plurality of flexible sizing elements being configured to be: extendable radially from the flexible shaft when in a deployed state; and at least partially foldable around a longitudinal axis of the flexible sizing elements when in a retracted state.

B. The valve planning tool of A, wherein the plurality of flexible sizing elements include a lengthwise profile having an outer edge with a first distance value from the longitudinal axis at a proximal end of the flexible sizing elements, a second distance value from the longitudinal axis at a distal end of the flexible sizing elements, and a third distance value from the longitudinal axis at a position between the proximal end and the distal end of the flexible sizing elements, wherein the third distance value is greater than the first and second distance values.

C. The valve planning tool of B, wherein the distance value of the outer edge from the longitudinal axis increases at a predefined rate from the first distance value to the third distance value, wherein the distance value of the outer edge from the longitudinal axis decreases at a predefined rate from the third distance value to the second distance value.

D. The valve planning tool of B, wherein at least a portion of the outer edge of at least one of the plurality of flexible sizing elements includes at least one of a beveled edge or a chamfered edge.

E. The valve planning tool of D, wherein the plurality of flexible sizing elements are configured to fold around the longitudinal axis based on the at least one beveled edge or chamfered edge.

F. The valve planning tool of B, wherein the third distance value represents a sealing dimension of a valve.

G. The valve planning tool of A, further comprising an anchor indicator configured to be coupable to the flexible shaft distal from the plurality of sizing elements.

H. The valve planning tool of G, further comprising a delivery catheter length simulator configured to extend distally from the anchor indicator.

I. A system comprising: a scope having a handle and an insertion tube with a working channel and a camera; an image processor configured to be in signal communication with the camera; a display device configured to be in signal communication with the image processor; and a valve planning tool comprising: a flexible shaft having a distal end and a proximal end; and a plurality of flexible sizing elements configured to be attachable to the flexible shaft, the plurality of flexible sizing elements being configured to be: extendable radially from the flexible shaft when in a deployed state; and at least partially foldable around a longitudinal axis of the flexible sizing elements when in a retracted state.

J. The system of I, wherein the plurality of flexible sizing elements include a lengthwise profile having an outer edge with a first distance value from the longitudinal axis at a proximal end of the flexible sizing elements, a second distance value from the longitudinal axis at a distal end of the flexible sizing elements, and a third distance value from the longitudinal axis at a position between the proximal end and the distal end of the flexible sizing elements, wherein the third distance value is greater than the first and second distance values.

K. The system of J, wherein at least a portion of the outer edge of at least one of the plurality of flexible sizing elements includes at least one of a beveled edge or a chamfered edge.

L. The valve planning tool of J, wherein the valve planning tool further includes: an anchor indicator configured to be coupable to the flexible shaft distal from the plurality of sizing elements; and a delivery catheter length simulator configured to extend distally from the anchor indicator.

M. A method comprising: providing a flexible shaft having a distal end and a proximal end; and attaching a plurality of flexible sizing elements to the flexible shaft, the plurality of flexible sizing elements being configured to be: extendable radially from the flexible shaft when in a deployed state; and at least partially foldable around a longitudinal axis of the flexible sizing element when in a retracted state.

N. The method of M, further comprising providing the flexible sizing elements with an outer edge having: a first distance value from the longitudinal axis at a proximal end of the flexible sizing elements; a second distance value from the longitudinal axis at a distal end of the flexible sizing elements; and a third distance value from the longitudinal axis at a position between the proximal end and the distal end of the flexible sizing elements, wherein the third distance value is greater than the first and second distance values.

O. The method of N, wherein the distance value of the outer edge from the longitudinal axis increases at a predefined rate from the first distance value to the third distance value, wherein the distance value of the outer edge from the longitudinal axis decreases at a predefined rate from the third distance value to the second distance value.

P. The method of N, further comprising providing at least a portion of the outer edge of at least one of the plurality of flexible sizing elements with at least one of a beveled edge or a chamfered edge.

Q. The method of P, further comprising at least partially folding the plurality of flexible sizing elements around the longitudinal axis based on the at least one beveled edge or chamfered edge.

R. The method of N, wherein the third distance value represents a sealing dimension of a valve.

S. The method of M, further comprising attaching an anchor indicator to the flexible shaft distal from the plurality of sizing elements.

T. The method of S, further comprising attaching a delivery catheter length simulator distally from the anchor indicator.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (for example “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. A valve planning tool comprising:
a flexible shaft having a distal end and a proximal end; and
a plurality of flexible sizing elements configured to be attachable to the flexible shaft, the plurality of flexible sizing elements being configured to be:
extendable radially from the flexible shaft when in a deployed state; and
at least partially foldable around a longitudinal axis of the flexible sizing elements when in a retracted state.

2. The valve planning tool of claim 1, wherein the plurality of flexible sizing elements include a lengthwise profile having an outer edge with a first distance value from the longitudinal axis at a proximal end of the flexible sizing elements, a second distance value from the longitudinal axis at a distal end of the flexible sizing elements, and a third distance value from the longitudinal axis at a position between the proximal end and the distal end of the flexible sizing elements, wherein the third distance value is greater than the first and second distance values.

3. The valve planning tool of claim 2, wherein the distance value of the outer edge from the longitudinal axis increases at a predefined rate from the first distance value to the third distance value,
wherein the distance value of the outer edge from the longitudinal axis decreases at a predefined rate from the third distance value to the second distance value.

4. The valve planning tool of claim 2, wherein at least a portion of the outer edge of at least one of the plurality of flexible sizing elements includes at least one of a beveled edge or a chamfered edge.

5. The valve planning tool of claim 4, wherein the plurality of flexible sizing elements are configured to fold around the longitudinal axis based on the at least one beveled edge or chamfered edge.

6. The valve planning tool of claim 2, wherein the third distance value represents a sealing dimension of a valve.

7. The valve planning tool of claim 1, further comprising an anchor indicator configured to be coupable to the flexible shaft distal from the plurality of sizing elements.

8. The valve planning tool of claim 7, further comprising a delivery catheter length simulator configured to extend distally from the anchor indicator.

9. A system comprising:
a scope having a handle and an insertion tube with a working channel and a camera;
an image processor configured to be in signal communication with the camera;
a display device configured to be in signal communication with the image processor; and
a valve planning tool comprising:
a flexible shaft having a distal end and a proximal end; and
a plurality of flexible sizing elements configured to be attachable to the flexible shaft, the plurality of flexible sizing elements being configured to be:
extendable radially from the flexible shaft when in a deployed state; and
at least partially foldable around a longitudinal axis of the flexible sizing elements when in a retracted state.

10. The system of claim 9, wherein the plurality of flexible sizing elements include a lengthwise profile having an outer edge with a first distance value from the longitudinal axis at a proximal end of the flexible sizing elements, a second distance value from the longitudinal axis at a distal end of the flexible sizing elements, and a third distance value from the longitudinal axis at a position between the proximal end and the distal end of the flexible sizing elements, wherein the third distance value is greater than the first and second distance values.

11. The system of claim 10, wherein at least a portion of the outer edge of at least one of the plurality of flexible sizing elements includes at least one of a beveled edge or a chamfered edge.

12. The valve planning tool of claim 2, wherein the valve planning tool further includes:
an anchor indicator configured to be coupable to the flexible shaft distal from the plurality of sizing elements; and
a delivery catheter length simulator configured to extend distally from the anchor indicator.

13. A method comprising:
providing a flexible shaft having a distal end and a proximal end; and
attaching a plurality of flexible sizing elements to the flexible shaft, the plurality of flexible sizing elements being configured to be:
extendable radially from the flexible shaft when in a deployed state; and
at least partially foldable around a longitudinal axis of the flexible sizing element when in a retracted state.

14. The method of claim 13, further comprising providing the flexible sizing elements with an outer edge having:
a first distance value from the longitudinal axis at a proximal end of the flexible sizing elements;
a second distance value from the longitudinal axis at a distal end of the flexible sizing elements; and
a third distance value from the longitudinal axis at a position between the proximal end and the distal end of the flexible sizing elements,
wherein the third distance value is greater than the first and second distance values.

15. The method of claim 14, wherein the distance value of the outer edge from the longitudinal axis increases at a predefined rate from the first distance value to the third distance value,
wherein the distance value of the outer edge from the longitudinal axis decreases at a predefined rate from the third distance value to the second distance value.

16. The method of claim 14, further comprising providing at least a portion of the outer edge of at least one of the plurality of flexible sizing elements with at least one of a beveled edge or a chamfered edge.

17. The method of claim 16, further comprising at least partially folding the plurality of flexible sizing elements around the longitudinal axis based on the at least one beveled edge or chamfered edge.

18. The method of claim 14, wherein the third distance value represents a sealing dimension of a valve.

19. The method of claim 13, further comprising attaching an anchor indicator to the flexible shaft distal from the plurality of sizing elements.

20. The method of claim 19, further comprising attaching a delivery catheter length simulator distally from the anchor indicator.
